(19) United States
(12) Patent Application Publication
(10) Pub. No.: US 2009/0104134 A1
(43) Pub. Date: Apr. 23, 2009

(54) ORGANOPHILIC CLAY FOR THICKENING ORGANIC SOLVENTS

(75) Inventors: Ashoke K. SenGupta, Barrington, IL (US); David McGregor, Grayslake, IL (US); Ralph Spindler, Palatine, IL (US); Kevin Cureton, Evanston, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: AMCOL International Corporation, Arlington Heights, IL (US)

(21) Appl. No.: 12/282,377

(22) PCT Filed: Jun. 6, 2007
(86) PCT No.: PCT/US07/13354

§ 371 Date: Sep. 10, 2008

Related U.S. Application Data
(60) Provisional application No. 60/804,137, filed on Jun. 7, 2006.

(51) Int. Cl. A61K 47/30; C08K 3/34; C08K 3/26; A61Q 1/04; A61Q 3/02; A61Q 17/04; A61Q 1/10; A61Q 11/00; C08K 3/22

(52) U.S. Cl. 424/59; 524/445; 524/425; 524/430; 524/432; 524/423; 524/431; 514/772.3; 424/63; 424/70.7; 424/64; 424/61

(57) ABSTRACT

An organophilic clay for thickening hydrophobic liquids comprising a layered silicate material, surfaces of said layered silicate material modified by an amphipathic copolymer prepared from a first comonomer that generates a hydrophilic homopolymer that is essentially insoluble in a hydrophobic liquid and a second comonomer that generates a hydrophobic homopolymer that is soluble in a hydrophobic liquid, said organophilic clay containing at least 10% by weight water, based on the dry weight of the layered silicate material.

### CROSS-REFERENCE TO RELATED APPLICATION

This application claims the benefit of U.S. Provisional application Ser. No. 60/804,137 filed on Jun. 7, 2006, which is a continuation-in-part of U.S. application Ser. No. 10/626,009 filed Jul. 24, 2003, hereby incorporated by reference which claims the benefit of U.S. provisional patent application Ser. No. 60/455,049 filed Mar. 14, 2003, and U.S. provisional patent application Ser. No. 60/398,631, filed Jul. 25, 2002.

### DESCRIPTION OF THE INVENTION

The present invention relates to an organophilic smectite clay that is capable of thickening organic solvents, in particular, ethyl acetate and butyl acetate, the organic solvents used commonly in producing nail lacquer compositions. Unlike the organophilic clays described in the art, the organophilic smectite clay of the present invention is necessarily produced by adsorbing an amine-free or a non-quaternary-ammonium compound onto the surface of the smectite clay. The amine-free or the non-quaternary-ammonium compound used for treating the smectite clay surface is essentially an amphiphilic copolymer. The amphiphilic copolymer useful for the object of the present invention is a block or a graft copolymer prepared from i) a hydrophilic comonomer that generates a homopolymer that is insoluble in the nail lacquer solvents, and ii) a hydrophobic comonomer that generates a homopolymer that is soluble in the nail lacquer solvents. Preferably, the mole fraction of the hydrophilic comonomer is in the range of 0.05-0.5, while the mole fraction of the hydrophobic comonomer is in the range of 0.5-0.95, with the molar ratio of the two comonomers being such that the copolymer is soluble and/or dispersible in the nail lacquer solvents, but is insoluble or merely dispersible in water. Most preferably, the amphiphilic copolymer is an A-B-A type of triblock copolymer, wherein the letter “A” represents a hydrophobic comonomer and the letter “B” represents a hydrophilic comonomer. According to a preferred embodiment, the amphiphilic copolymer should have a weight average molecular weight (Mw) of at least 1,000 Dalton. Presently, the most preferred amphiphilic copolymer for producing the organophilic smectite clay of the present invention is a triblock copolymer, PEG 30 Dipolyhydroxystearate, available from Uniqema under the tradename of Arlacel P-135.

The organophilic smectite clay of the present invention is produced in accordance with a procedure comprising of the following sequential steps:

i) The smectite clay is dispersed in water under high-shear mixing, using, for example, a high-shear homogenizer or a colloid mill, preferably while maintaining the temperature of the dispersion in the range of 15-75° C. The clay content (based on dry or moisture-free clay) of the dispersion is preferably in the range of 0.5-75%, and most preferably in the range of 2-10% by weight, based on the total weight of clay and water. The dispersion is subjected to high-shear mixing until the particle size of the sheared clay particles is less than the particle size of the unsheared clay particles by an amount preferably ranging from about 10 to about 80%, and most preferably from about 30 to about 50%.

ii) Upon obtaining the aforementioned particle size reduction via shearing the clay dispersion, an amphiphilic copolymer of the type described above is mixed into the clay dispersion and the resulting clay dispersion composition is subjected to further shearing until at least about 2.5% by weight of the added amphiphilic polymer is adsorbed on the clay surface, while maintaining the dispersion-temperature in the range of 35-60° C. The weight of the amphiphilic copolymer added to the clay dispersion is preferably in the range of 10-200%, and most preferably in the range of 50-100%, based on the weight of the smectite clay (dry-basis).

iii) The clay dispersion composition from step (ii) is subjected to drying using methods or unit operations known in the art, in order to obtain a final moisture-content ranging from about 5 to about 20%, based on the total weight of the final organophilic clay composition.

iv) The dried organophilic clay gallant is typically ground to a particle size in the range of 0.1-5,000 microns, using grinding equipments known in the art.

Alternatively, the organophilic clay composition may be produced by mixing a smectite with a mixture of water and an amphiphilic copolymer of the type described in a preceding section, using a high-shear mixer, wherein the organophilic clay composition conforms to the following ingredient proportions by weight:

Smectite clay: 10-55%

### Amphiphilic Copolymer: 5-110%

### Water: 1.5-20%

The ability of the organophilic clay of the present invention for thickening nail lacquer solvents, namely, ethyl acetate and butyl acetate, is demonstrated as per the data presented in Table I. To illustrate further the benefit of the present invention, Table I also includes viscosity data for a quaternary ammonium compound-modified organoclay known in the art. The method used for conducting the viscosity tests whose results are presented in Table I is as follows.

The organophilic clay of the present invention was prepared by mixing a smectite clay with PEG 30 Dipolyhydroxystearate (amphiphilic copolymer), as per the manufacturing steps described above. Prior to step (iii) involving drying of clay dispersion, the clay dispersion contained 7.52% (dry-basis—8.25% on wet basis) of the smectite clay, 8.25% of the amphiphilic copolymer, and 83.5% of water (excluding the water associated with the moist clay). The dried and ground organophilic clay produced finally had a moisture content of about 13.5% by weight of the whole organophilic clay. A butyl acetate-mastergel (pregel) of the dried organophilic clay was prepared by shearing the organoclay in butyl acetate using a rotor-stator homogenizer (Silverson Homogenizer). Subsequently, the mastergel was diluted in each of ethyl acetate and butyl acetate, by mixing the mastergel into the respective solvents, using a dispersion-blade agitator. The clay-content (dry and surface modifier-free basis) of the diluted compositions was about 3% by weight. The diluted compositions were stored for a period of one hour before they were taken for Brookfield viscosity measurements.

Likewise, a butyl acetate-mastergel of a quaternary ammonium compound-modified organoclay (Tradename: Bentone 27V obtained from Elementis) was prepared. The organoclay mastergel was diluted in each of the aforementioned solvents to a final clay-content (dry and surface modifier-free basis) of about 3% by weight, wherein two polar compounds (commonly referred to as polar activators in the organoclay prior art), propylene carbonate and water, had to be added in order to produce thickening in the dilute compositions (the compositions hardly thickened when either of the two polar activators was added singly). The amount of water added as a polar activator was about 27% by weight, based on the weight of clay (dry and surface modifier-free basis). The amount of propylene carbonate added as a polar activator was about 30% by weight, based on the weight of clay (dry and surface modifier-free basis), an amount typically used in the art.

TABLE 1

| Organophilic | | | | | | |
|---|---|---|---|---|---|---|
| Clay, | | | | | | |
| Weight % of | | Brookfield RVT Viscosity, cps | | | | |
| Smectite Clay | Solvent | 0.5 rpm | 1 rpm | 5 rpm | 10 rpm | 20 rpm |
| Present | Ethyl | 120,000 | 60,000 | 15,200 | 8,000 | 3,500 |
| Invention, 3% | Acetate | | | | | |
| Present | Butyl | 48,000 | 30,000 | 6,000 | 2,400 | 2,000 |
| Invention, 3% | Acetate | | | | | |
| Quaternary | Ethyl | 8,000 | 4,000 | 2,320 | 1,240 | 720 |
| Ammonium | Acetate | | | | | |
| Compound- | | | | | | |
| modified | | | | | | |
| Organoclay | | | | | | |
| (Bentone 27V), | | | | | | |
| 3% | | | | | | |
| Quaternary | Butyl | 6,400 | 3,200 | 800 | 500 | 200 |
| Ammonium | Acetate | | | | | |
| Compound- | | | | | | |
| modified | | | | | | |
| Organoclay | | | | | | |
| (Bentone 27V), | | | | | | |
| 3% | | | | | | |

### METHOD OF MANUFACTURE

Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

It has been found that excellent organic liquid thickening is achieved by adding water to the layered silicate material and shearing the layered silicate/water combination prior to adding the copolymer to the layered silicate/water composition, and thereafter continuing to mix the copolymer uniformly throughout the layered silicate/water combination. Alternatively, the water and copolymer can both be added to the layered silicate material and the composition sheared until homogeneous. When the layered silicate/water copolymer composition is homogeneous, the composition then is dried to a moisture content in the range of about 5% to about 20% by weight, based on the total weight of the composition.

The water content of the layered silicate/water/copolymer composition, when sheared to provide a homogeneous blend, should be at least 5% by weight based on the dry weight of the layered silicate material, preferably at least 10% by weight, more preferably at least 20% by weight water, based on the dry weight of the layered silicate material. Any higher amount of water, e.g., a layered silicate slurry having 200-500% water, can be used but is not economical since the composition is then dried to a moisture content of about 5% to about 20%, preferably about 10% to about 15% water, based on the dry weight of the layered silicate material, prior to adding the composition to one or more organic/liquid(s), e.g., film formers, for increasing the viscosity of the liquid(s). The final product (organophilic clay containing the amphipathic copolymer) should have a water content of at least 10% by weight, preferably 10% to 20% by weight water, based on the dry weight of the layered silicate material, to be effective in viscosifying organic liquids. The water content can be increased by adding water to the organophilic clay just prior to use if it has dried below 10% by weight prior to use.

The amount of copolymer contained in the organoclay should be at least about 10% by weight, based on the dry weight of the layered silicate material, preferably about 15% to about 150%, more preferably about 25% to about 100%, based on the dry weight of the layered silicate material.

What is claimed:

1. An organophilic clay for thickening hydrophobic liquids comprising a layered silicate material, surfaces of said layered silicate material modified by an amphipathic copolymer prepared from a first comonomer that generates a hydrophilic homopolymer that is essentially insoluble in a hydrophobic liquid and a second comonomer that generates a hydrophobic homopolymer that is soluble in a hydrophobic liquid, said organophilic clay containing at least 10% by weight water, based on the dry weight of the layered silicate material.

2. The clay of claim 1 further comprising a thickening aid.

3. The clay of claim 2 wherein the thickening aid is selected from the group consisting of propylene carbonate, hexylene glycol, ethanol, propylene glycol, butylene glycol, water, and mixtures thereof.

4. The clay of claim 1 wherein the hydrophobic liquid comprises one or more nonpolar liquid having a dielectric constant of less than about 10.

5. The clay of claim 1 wherein the hydrophobic liquid is selected from the group consisting of a silicone oil, a mineral oil, a liquid hydrocarbon, a petroleum-derived oil, an ester solvent, a vegetable oil, a flower oil, and mixtures thereof.

6. The clay of claim 1 wherein the layered silicate material comprises a smectite clay, a sodium lithium magnesium silicate, or a mixture thereof.

7. The clay of claim 6 wherein the smectite clay is selected from the group consisting of bentonite, montmorillonite, saponite, hectorite, bidelite, stevensite, and mixtures thereof.

8. The clay of claim 1 wherein the copolymer is a graft copolymer or a block copolymer.

9. The clay of claim 1 wherein the copolymer is soluble or dispersible in hydrophobic liquids having a dielectric constant of less than about 10.

10. The clay of claim 9 wherein the copolymer comprises a triblock copolymer.

11. The clay of claim 10 wherein the triblock copolymer comprises poly(ethylene glycol-30)-co-dipoly(hydroxystearate), BIS PEG 15 dimethicone/IPDI copolymer, or a mixture thereof.

12. The clay of claim 1 wherein the first comonomer, when polymerized, provides a homopolymer selected from the group consisting of poly(oxyethylene), poly(ethylene glycol), poly(propylene glycol), poly(vinyl chloride), poly(acrylate), and poly(acrylamide).

13. The clay of claim 1 wherein the second comonomer, when polymerized, provides a homopolymer selected from the group consisting of poly(hydroxystearate), poly(12-hydroxystearic acid), poly)lauryl methacrylate), polystyrene, poly(dimethylsiloxane), poly(vinyl acetate), poly(methyl methacrylate), and poly(vinyl methyl ether).

14. The clay of claim 1 comprising about 30% to about 90% of the hydrophobic liquid, about 0.5% to about 70% of the layered silicate, and about 0.025% to about 50% of the copolymer, by weight, of the composition.

15. The clay of claim 14 further comprising a thickening aid in an amount of about 0.025% to about 20%, by weight, of the composition.

16. The clay of claim 1 further comprising about 0.1% to about 50%, by weight, of the composition of at least one functional particulate material.

17. The clay of claim 16 wherein the functional particulate material is selected from the group consisting of titanium dioxide, mica, calcium carbonate, kaolinite clay, alumina, talc, zinc oxide, calcium sulfate, iron oxide, an organic pigment, and mixtures thereof.

18. The organophilic clay of claim 1, wherein the amphipathic copolymer comprises poly(ethylene glycol-30)-co-dipoly-(hydroxystearate), polydimethylsiloxane-polyoxyethylene 15 polymer/3-isocyanatomethyl-3,5,5-trimethylcyclohexyl isocyanate copolymer, or a mixture thereof.

19. A method of manufacturing the organophilic clay of claim 1 comprising shearing a composition comprising the layered silicate material, water, and the amphipathic copolymer together, wherein the water content is at least 10% by weight, based on the dry weight of the layered silicate material, until the composition is homogeneous; and drying the composition to a water content of at least 10% by weight, based on the dry weight of the layered silicate material.

20. The method of claim 19, wherein the layered silicate is sheared with the water prior to adding the copolymer, and thereafter adding the copolymer and continuing to shear until the composition is homogeneous.

21. The method of claim 20, wherein the water content during shearing is in the range of 10-200%, based on the dry weight of the layered silicate material.

22. The method of claim 21, wherein the amount of copolymer added to the layered silicate and water is about 25% to about 100%, based on the dry weight of the layered silicate material.

23. A method of thickening a hydrophobic composition comprising adding a sufficient amount of the organophilic clay of claim 1 to the hydrophobic composition to provide increased viscosity.

24. The method of claim 23 wherein the hydrophobic composition is selected from the group consisting of a cosmetic product, a personal care product, and a pharmaceutical product.

25. The method of claim 23 wherein the hydrophobic composition is selected from the group consisting of a liquid makeup, an eye shadow, a mascara, a lip color, a nail polish, an antiperspirant, a deodorant, a pharmaceutical product, a sunscreen, a paint, and a coating product.

26. A method of dispersing a particulate material in a hydrophobic solvent comprising adding the particulate material to the hydrophobic solvent, and adding a sufficient amount of the organophilic clay of claim 1 to the hydrophobic solvent to disperse and suspend the particulate material in the hydrophobic solvent.

27. The method of claim 26 wherein the particulate material is selected from the group consisting of titanium dioxide, calcium carbonate, kaolinite clay, alumina, talc, zinc oxide, calcium sulfate, an organic pigment, iron oxide, and mixtures thereof.
